(19) United States
(12) Patent Application Publication
(10) Pub. No.: US 2025/0276122 A1
(43) Pub. Date: Sep. 4, 2025

(54) Nasal Aspirator Capable of Automatically Relieving Pressure and Method for Sucking Nasal Mucus

(71) Applicant: Haoyu PAN, Guangxi (CN)

(72) Inventor: Haoyu PAN, Guangxi (CN)

(21) Appl. No.: 18/593,946

(22) Filed: Mar. 3, 2024

(51) Int. Cl. A61M 1/00

(52) U.S. Cl. CPC A61M 1/68; A61M 1/82; A61M 2205/502

(57) ABSTRACT

A nasal aspirator capable of automatically relieving pressure comprises casing, air pump component, air bag, constant pressure device and suction component; wherein, the air pump component is communicated with the air bag, and the air bag is communicated with the constant pressure device; when the air pump component works, negative pressure will be generated in the air bag; when the pressure exceeds a threshold, a pressure releasing valve will be opened to keep the air pressure inside the air bag stable; when the pressure is less than the threshold, the pressure releasing valve will be closed; and the suction component is communicated with the air bag; it further comprises a switch component, wherein the suction component defines a path for external air to enter the air bag, and the suction component is also provided with a pressure releasing channel, and the switch component can open or close the channel.

### TECHNICAL FIELD

The present invention relates to the technical field of nasal aspirators, in particular to a nasal aspirator capable of automatically relieving pressure and a nasal mucus suction method.

### BACKGROUND

Nasal aspirators are usually used to clean the nostrils of people or any other animals. For example, infants and young children lack the ability to clean their nostrils efficiently, and nasal congestion will last for a long time, affecting breathing, eating, breastfeeding, sleep and mood. The nasal aspirator can help parents to discharge secretions from the nasal cavity, so that infants can breathe smoothly without being affected by nasal congestion. Therefore, parents may buy various types and functions of nose inhalers. A common nasal aspirator is a rubber ball. The ball has a tip that can be inserted into the baby's nostril. Squeezing the ball can air exhaust the air in the ball through the top end. When the ball is released, a low pressure area (negative pressure area) will be formed in the ball, and air (and possibly mucus) will be brought into the ball through the tip. When the ball is inflated again due to elasticity, the pressure will reach a balance (return to the natural state). This process will generate a suction force from the tip of the nose to suck out the secretions in the nostril (assuming that the tip is placed in the nostril when it is released), but it is not easy to suck out the secretions in the nasal cavity of infants, and parents cannot control the strength when using it, which is very likely to hurt the nasal cavity of infants.

For example, the patent document U.S. Pat. No. 9,993,584 “Nasal Aspirator” discloses a suction device. A described nasal aspirator system can have accurate, fine-tuned control of suction through the nasal aspirator system. The nasal aspirator system can include a tip inserted into a baby's nostril, with another end inserted into a parent's mouth. The parent can have accurate control of an amount of suction by regulating their own lungs and depth and speed of breath. The described nasal aspirator system is therefore much safer than previous aspirator systems since a parent can precisely control the suction and stop instantaneously (as required). However, this kind of nasal aspirator is more traditional, needs manual control, and cannot control the efficiency, and the user experience is not good enough.

Another kind of nasal aspirator usually includes a suction head and a bulb body part, the bulb body part is provided with an air pump, the suction head is provided with a cavity, and the cavity wall opening of the cavity forms a suction port. When the dirt is sucked into the cavity, it is easy to block the suction port, and then the dirt is sucked into the main machine such as the air pump, which is easy to cause damage to the main machine.

For example, the patent document U.S. Pat. No. 11,400,200 “DEVICE FOR AUTOMATICALLY COLLECTING SNOT” discloses a device for automatically collecting snot, which comprises a suction nozzle, a collecting cup, a cup holder and a casing component which are detachably connected in sequence, wherein the casing component is a hollow cavity. The present disclosure generates negative pressure suction through the air pump, and then the suction air enters the inner collecting cup through the air suction pipe, the cup holder air suction port, the air suction port channel and the micropore in sequence. Finally, the suction sucks the snot in the nose of a user into the inner collecting cup through the suction nozzle. An air suction port channel is arranged opposite to the air suction port of the cup holder. The flow direction of the air channel changes. With the anti-reflux function of the cup cover, the problem that the nose is sucked back into the casing component and damages the host machine is effectively solved. Meanwhile, the cup cover is provided with micropores. This design can not only ensure the normal air intake effect, but also make use of the negative pressure of micropores to further prevent the nose from flowing backwards in the inner collecting cup. However, although the device for automatically collecting nasal mucus solves the problem that the nasal mucus is sucked back into the casing component and damages the host computer, the device is not controlled by a constant pressure device, and the suction intensity of negative pressure generated by the air pump cannot be adjusted, so that the nasal cavity of infants may be injured when being used.

The nasal aspirator method disclosed in the patent document US 20150093268 “FOOT OPERATED SNIVEL SUCTION DEVICE” solves the problem that the negative pressure suction force cannot be adjusted. This document discloses a nasal aspirator, which comprises a base, two pumps, two pistons, two piston rods, a pedal and two casings. A pressure releasing valve is fixed in the casing with an air suction hole, and is connected between the air suction manifold and one of the air suction pipes. When the air suction pressure is too high, which may damage the nasal mucosa, the pressure releasing valve releases the pressure, and the sound generator sends out a warning sound, prompting the user to adjust the air suction pressure or check whether there is any wrong operation, so as to protect the user. Although this kind of pedal nasal aspirator solves the problem of air pump pressure, its structure is more complicated, and it still needs manual control for the change from the most traditional hand extrusion to pedal. Moreover, the air releasing valve deflates and discharges pressure after the pressure is higher than the normal pressure value, and it is not controlled at a stable pressure value, which still has certain safety risks during use.

### SUMMARY

Accordingly, the present invention aims to provide a nasal aspirator that can automatically relieve pressure, so as to improve the above shortcomings.

The present invention provides a nasal aspirator, including:

a casing with an accommodating cavity, an air pump component, an air bag, a

constant pressure device and a suction component arranged in the accommodating cavity; and

wherein, the air pump component is communicated with the air bag and comprises at least one air flow connector, and the air flow connector defines an air suction channel and an air exhaust channel of the air pump component; and

wherein the air bag comprises at least one air chamber, and at least one communication member is arranged in the air chamber, and the communication member is configured to receive the air flow connector; and

wherein, the constant pressure device is arranged on the air bag and communicated with the air chamber, and comprises at least one pressure releasing valve; and the pressure releasing valve provides an overflow channel for air in the air chamber when the air in the air chamber exceeds a predetermined threshold; and

wherein the suction component is communicated with the air bag, and the suction component defines a path for external air to enter the air bag.

The present invention provides another nasal aspirator capable of automatically relieving pressure, which includes:

a casing with an accommodating cavity, an air pump component, an air bag, a constant pressure device and a suction component arranged in the accommodating cavity; and

wherein, the air pump component is communicated with the air bag and comprises at least one air flow connector, and the air flow connector defines an air suction channel and an air exhaust channel of the air pump component; and

wherein the air bag comprises at least one air chamber, and at least one communication member is arranged in the air chamber, and the communication member is configured to receive the air flow connector; and

wherein, the constant pressure device is arranged on the air bag and communicated with the air chamber, and comprises at least one pressure releasing valve; the pressure releasing valve provides an overflow channel for air in the air chamber when the air in the air chamber exceeds a predetermined threshold; and

wherein, the suction component defines an airflow path for external air to enter the air bag, and comprises a suction nozzle, a storage compartment and a hose, wherein the storage compartment is arranged between the suction nozzle and the hose, the storage compartment is configured to contain nasal mucus, and the hose is communicated with the air bag; and

wherein the storage compartment further comprises a pressure releasing channel and a switch component, and the switch component can open and close the pressure releasing channel.

The present invention also provides a method for sucking nasal mucus, which includes the following steps: providing a nasal aspirator capable of automatically relieving pressure, wherein the nasal aspirator comprises a casing with an accommodating cavity, an air pump component, an air bag, a constant pressure device and a suction component arranged in the accommodating cavity; and wherein, the air pump component is communicated with the air bag and comprises at least one air flow connector, and the air flow connector defines an air suction channel and an air exhaust channel of the air pump component; and wherein the air bag comprises at least one air chamber, at least one communication member is arranged in the air chamber, and the communication member is configured to receive the air flow connector; and wherein the constant pressure device is arranged on the air bag and communicated with the air chamber, and comprises at least one pressure releasing valve, and the pressure releasing valve provides an overflow channel for air in the air chamber when the air in the air chamber exceeds a predetermined threshold; and wherein the suction component is communicated with the air bag and comprises a suction nozzle and a storage compartment, and the suction component defines a path for external air to enter the air bag; and

opening the air pump component;

placing the suction nozzle close to a source of nasal mucus;

absorbing the nasal mucus and storing the nasal mucus in the storage compartment; and

closing the air pump component after absorbing the nasal mucus.

### BRIEF DESCRIPTION OF DRAWINGS

In order to explain the technical solution of this application more clearly, the drawings needed in the implementation will be briefly introduced below. Obviously, the drawings described below are only some implementations of this application. For those skilled in the art, other drawings can be obtained according to these drawings without creative work.

FIG. 1 is an overall view of a nasal aspirator capable of automatically relieving pressure;

FIG. 2 is a cross-sectional view of the back of the casing of the nasal aspirator capable of automatically relieving pressure;

FIG. 3 is a partial view of the air pump component, air bag and constant pressure device in the nasal aspirator capable of automatically relieving pressure;

FIG. 4 is an exploded view of the front of the casing of the nasal aspirator capable of automatically relieving pressure;

FIG. 5 is a front view of the casing of the nasal aspirator capable of automatically relieving pressure;

FIG. 6 is a partial view of the nasal aspirator capable of automatically relieving pressure, including a hose and an air suction component;

FIG. 7 is a cross-sectional view of the lower compartment of the nasal aspirator capable of automatically relieving pressure;

FIG. 8 is a schematic view of the suction component in the nasal aspirator capable of automatically relieving pressure.

In the figures: Casing (1000); Music playing component (1300); Lighting component (1400); Electronic control module (1500); Control circuit board (1510); Control button (1520); Power key (1521); Light key (1522); Music playing key (1523); Gear reduction key (1524); Gear increase key (1525); display panel (1600); Battery component (1700); Charging hole (1800); Air pump component (2000); Airflow connector (2001); Air suction connector (2100); Air exhaust connector (2200); Air bag (3000); Air chamber (3001); Air suction chamber (3100); Air suction port (3110); Suction communication member (3120); Air exhaust chamber (3200); A communication member: (3002); An air exhaust port (3210); An air exhaust Communication member (3220); Constant pressure device (4000); Pressure releasing valve (4100); Hose (5000); Suction component (6000); Storage compartment (6100); Upper compartment (6110); Suction nozzle mounting seat (6111); Lower compartment (6120); Slot (6121); Sealing ring (6122); Mounting seat (6123); Upper mounting seat (6124); Lower mounting seat (6125); Pressure releasing channel (6201); Switch component (6200); Suction nozzle (6300); Bracket (6400); First cover plate (6410); Second cover plate (6420); Through hole (6430); Airflow pipe (6440); Accommodating cavity (7000).

### DESCRIPTION OF EMBODIMENTS

In describing the preferred embodiments, specific terminology will be resorted to for the sake of clarity. It is to be understood that each specific term includes all technical equivalents which operate in a similar manner to accomplish a similar purpose.

While various aspects and features of certain embodiments have been summarized above, the following detailed description illustrates a few exemplary embodiments in further detail to enable one skilled in the art to practice such embodiments. Reference will now be made in detail to embodiments of the inventive concept, examples of which are illustrated in the accompanying drawings. The accompanying drawings are not necessarily drawn to scale. The described examples are provided for illustrative purposes and are not intended to limit the scope of the invention. It should be understood, however, that persons having ordinary skill in the art may practice the inventive concept without these specific details.

It will be understood that, although the terms first, second, etc. may be used herein to describe various elements, these elements should not be limited by these terms. These terms are only used to distinguish one element from another. For example, a first attachment could be termed a second attachment, and, similarly, a second attachment could be termed a first attachment, without departing from the scope of the inventive concept.

It will be understood that when an element or layer is referred to as being “on,” “coupled to,” or “connected to” another element or layer, it can be directly on, directly coupled to or directly connected to the other element or layer, or intervening elements or layers may be present. In contrast, when an element is referred to as being “directly on,” “directly coupled to,” or “directly connected to” another element or layer, there are no intervening elements or layers present. Like numbers refer to like elements throughout. As used herein, the term “and/or” includes any and all combinations of one or more of the associated listed items.

As used in the description of the inventive concept and the appended claims, the singular forms “a,” “an,” and “the” are intended to include the plural forms as well, unless the context clearly indicates other.

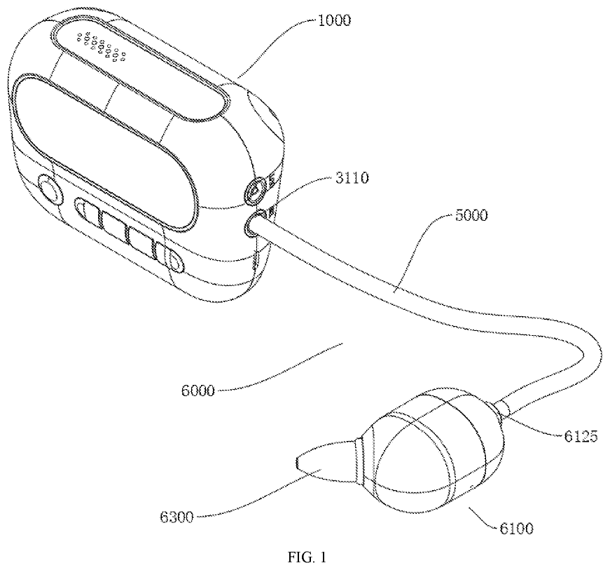

FIGS. 1 to 8 show a structure provided by a first embodiment of the present invention, and only parts related to the embodiment of the present invention are shown for convenience of explanation.

Referring to FIG. 1, FIG. 1 discloses the general structure of the present invention. The nasal aspirator capable of automatically relieving pressure includes a casing 1000 with an accommodating cavity 7000, and an air suction component 6000, which includes a hose 5000 providing an air channel, a storage compartment 6100 and a suction nozzle 6300. One end of the hose 5000 is detachably connected with the air suction port, and the other end is detachably connected with the lower mounting seat 6125, and the hose 5000, the air suction component 6000 and the air suction port are communicated.

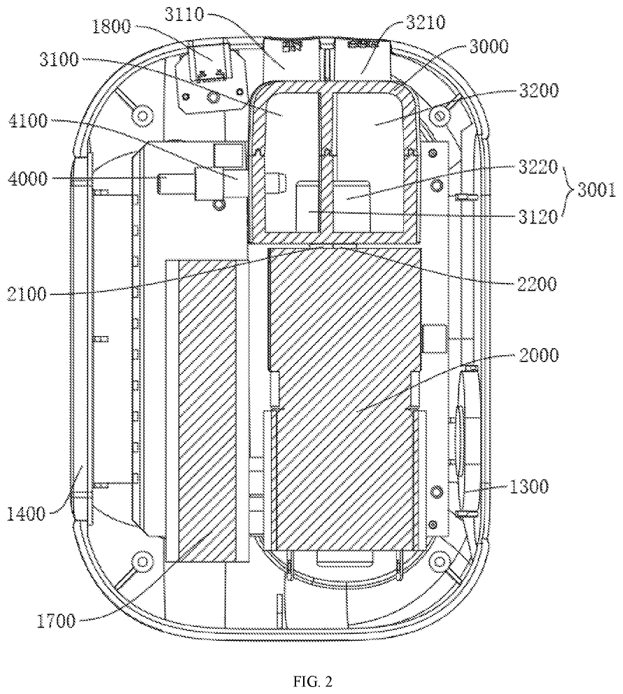

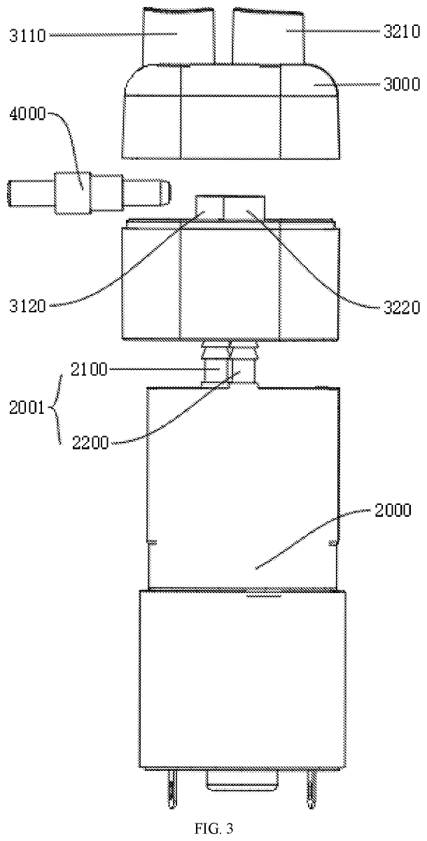

Referring to FIG. 2 and FIG. 3, an air pump component 2000 for providing negative pressure is arranged in the accommodating cavity 7000, and the nasal aspirator capable of automatically releasing pressure can perform suction and air exhaust actions through the air pump component 2000, and the air pump component 2000 is provided with an air flow connector 2001, which includes an air suction connector 2100 and an air exhaust connector 2200. In this embodiment, the accommodating cavity 7000 is also provided with an air bag 3000 connected with the air pump component 2000, and the air bag 3000 is composed of an air suction chamber 3100 and an air exhaust chamber 3200 which are independent from each other. The air bag 3000 is provided with an air suction port 3110 and an air suction communication member 3120 communicating with the air suction chamber 3100, an air exhaust port 3210 and an air exhaust communication member 3220 communicating with the air exhaust chamber 3200. The suction connector 2100 of the air pump component 2000 is fixed on the air suction communication member 3120 and is in air communication with the air suction chamber 3100, and the air exhaust connector 2200 of the air pump component 2000 is fixed on the air exhaust communication member 3220 and is in air communication with the air exhaust chamber 3200.

When the nasal aspirator capable of automatically relieving pressure starts to work, the air pump component 2000 generates negative pressure, and is communicated with the outside through the air suction connector 2100, the first connecting member, the air suction chamber 3100 and the air suction port 3110 in turn.

A constant pressure device 4000 for controlling the negative pressure is also arranged in the accommodating cavity 7000, and air flows between the constant pressure device 4000 and the air suction chamber 3100. A pressure releasing valve 4100 is arranged in the constant pressure device 4000, and the pressure releasing valve 4100 is opened when the pressure in the inhaled chamber 3100 reaches a threshold; when the pressure in the suction chamber 3100 is lower than the threshold, the pressure releasing valve 4100 is closed.

A lighting component 1400 is provided at one side of the accommodation cavity 7000 in the longitudinal direction, which can provide lighting function. When it is needed to be used at night, the lighting in the room will feel dazzling, especially for infants. The lighting component 1400 can provide lights with different intensities, thus solving the above problems.

A music playing device is provided at the other side of the accommodating cavity 7000 in the length direction relative to the lighting component 1400, and when in use, the music playing device can play, for example, gentle folk songs to attract the attention of infants, thus better completing the nose suction work.

There is also a battery module 1700 in the accommodating cavity 7000. The battery module 1700 provides the working power of the nasal aspirator which can automatically relieve pressure, and the device is charged through a charging hole 1800 which is located under the opening 1100 of the casing 1000. The charging hole 1800 can be a USB interface, a Type-c interface, a Lightning interface or other interfaces.

In another embodiment, the nasal aspirator is connected with a socket for power supply through an electrical connection device, such as an electrical plug or the like (not shown in the figure).

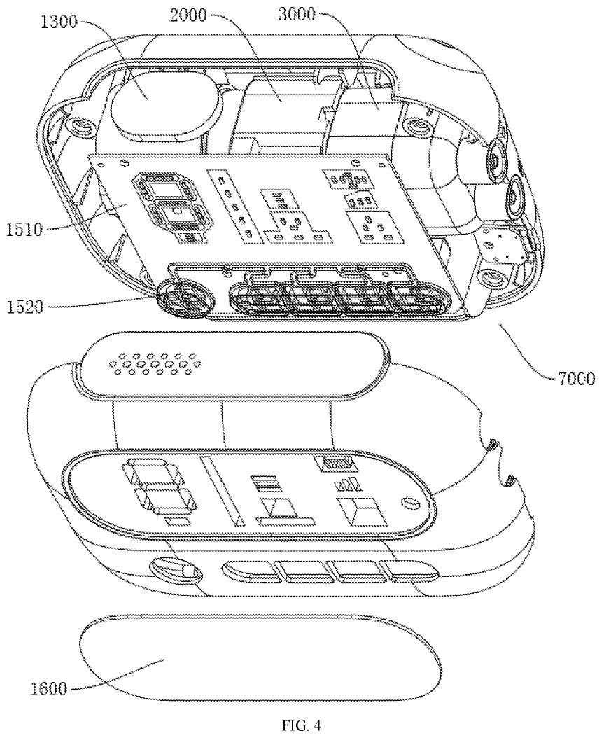

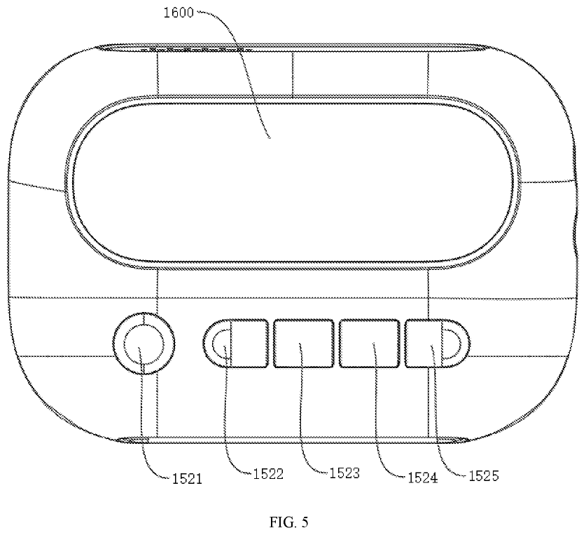

Referring to FIGS. 4 and 5, FIG. 4 shows the front and internal structure of the casing 1000 of the nasal aspirator capable of automatically relieving pressure, and FIG. 5 shows the control button of the nasal aspirator capable of automatically relieving pressure.

As shown in FIG. 4, an electronic control module 1500 is arranged in the accommodating cavity 7000, which includes a control circuit board 1510 and a control button 1520. The control circuit board 1510 is arranged in the accommodating cavity 7000, and the control circuit board 1510 is electrically connected to the air pump component 2000, the music playing component 1300 and the lighting component 1400, and the control button 1520 is arranged on the control circuit board 1510, penetrating the front of the casing 1000.

The suction generated by the nasal aspirator capable of automatically relieving pressure is divided into 9 gears, which can be adjusted by the control button 1520.

As shown in FIG. 5, the control button 1520 includes: a power key 1521, a light key 1522, a music playing key 1523, a gear reduction key 1524 and a gear increase key 1525. In this embodiment, the power key 1521 is long-pressed to turn on and off the machine, and the default suction gear position is the 3rd gear; the gear increase key 1525 is short-pressed to increase the gear, and the highest gear is the 9th gear; the gear reduction key 1524 is short-pressed to decrease the gear, and the minimum gear is the 1st gear; the power key 1521 is short-pressed to pause; the music playing key 1523 is short-pressed to cycle between low volume, medium volume, high volume and off. The light key is long-pressed to turn on or off the light, and is short-pressed to cycle between gear increase and gear reduction of the light intensity.

Before use, the nasal aspirator can be turned on, and the suction force of the suction nozzle 6300 can be felt by the palm before performing the nose suction work.

The front of the casing 1000 is also provided with a display panel 1600, which is connected to the electronic control module 1500. Through the display panel 1600, the current working state of the nasal aspirator capable of automatically relieving pressure can be known, for example gear, power situation, whether the lights are on, light intensity, whether the music is on and volume.

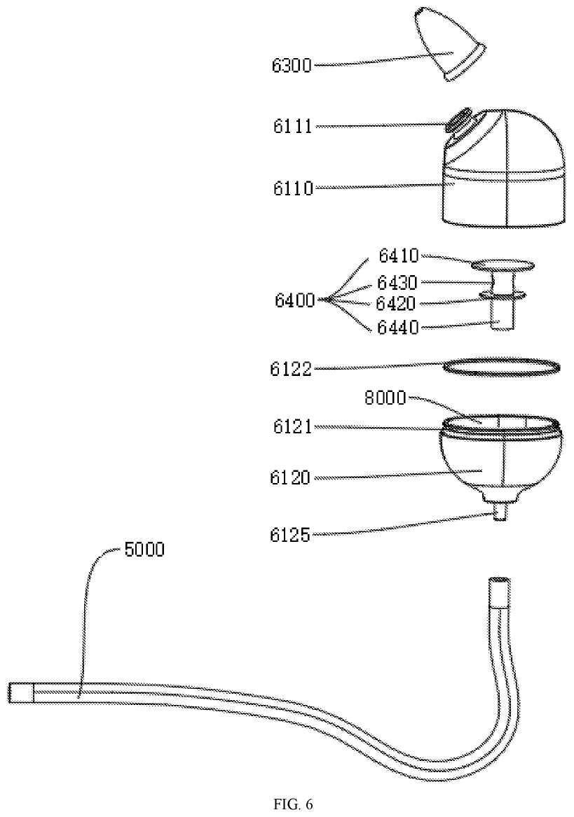

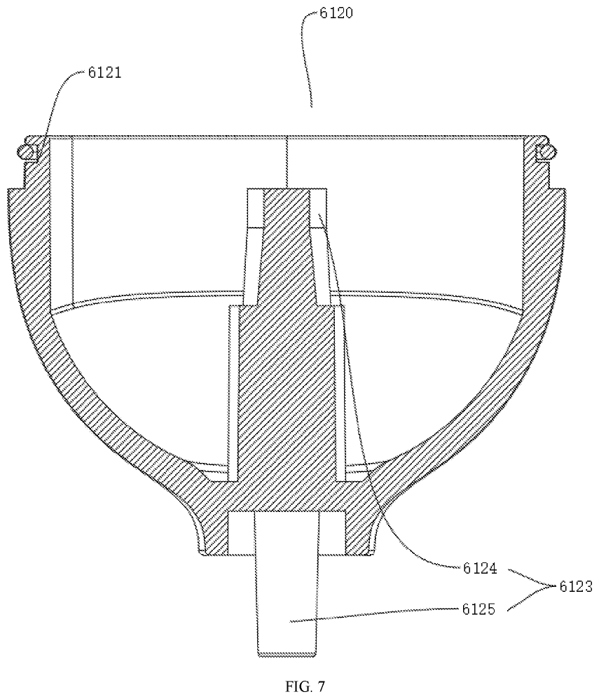

As shown in FIGS. 6 and 7, FIG. 6 shows the hose 5000 that can automatically relieve pressure and part of the suction component 6000. FIG. 7 shows the cross section of the lower compartment 6120. As shown in FIGS. 6 and 7, the air suction component 6000 includes a storage compartment 6100 with a cavity 8000, a suction nozzle 6300 fixed on the storage compartment 6100 and in air communication with the storage compartment 6100, and a bracket 6400 detachably installed in the cavity 8000.

The storage compartment 6100 is divided into an upper compartment 6110 and a lower compartment 6120. The lower compartment 6120 is provided with a first mounting seat 6123, which consists of an upper mounting seat 6124 and a lower mounting seat 6125. The hose 5000 is connected with the lower compartment 6120 through the lower mounting seat 6125, and the bracket 6400 is fixed with the lower compartment 6120 through the upper mounting seat 6124.

The lower compartment 6120 is provided with a slot 6121, and the slot 6121 is used for installing a sealing ring 6122. When the upper compartment 6110 and the lower compartment 6120 are connected, the sealing ring 6122 can prevent nasal secretions sucked into the storage compartment 6100 from flowing out, and thus there is no need to worry about secretions overflowing or affecting the surrounding environment. The sealing ring 6122 is usually made of soft and durable materials to ensure its effectiveness and long-term use. It is precisely installed in the 6121 and closely adheres to the storage compartment 6100, forming a reliable barrier to prevent any liquid or air from overflowing.

The bracket 6400 is provided with a first cover plate 6410, which closes the end of the airflow pipe 6440 far from the upper mounting seat 6124, and a second cover plate 6420 is provided below the first cover plate 6410. The first cover plate 6410 and the second cover plate 6420 are both circular, and the diameter of the first cover plate 6410 is larger than that of the second cover plate 6420. A through hole 6430 is opened between the first cover plate 6410 and the second cover plate 6420 for air communication between the storage compartment 6100 and the hose 5000. The first cover plate 6410 is used to prevent the sucked nasal secretions from being sucked into the hose 5000. Because the diameter of the first cover plate 6410 is larger than that of the second cover plate 6420, when the nasal aspirator capable of automatically relieving pressure works, the sucked nasal secretions will not fall on the second cover plate 6420 when they fall on the first cover plate 6410 and disperse around, but fall on the lower end of the storage compartment 6100, and the second cover plate 6420 will also prevent the nasal secretions sucked into the storage compartment 6100 from entering the through hole 6430 from below by suction.

At least a part of the storage compartment 6100 is made of a transparent material, and users can directly observe the internal situation of the storage compartment 6100 outside the storage compartment 6100 without opening or moving articles, and the storage compartment constructed of a transparent material provides visual convenience.

Suction nozzle 6300 can be made of medical grade silica gel, which ensures the safety and reliability of the product, does not contain any harmful substances, is non-toxic and tasteless, and meets the international hygiene standards through strict production technology and testing standards. The suction nozzle 6300 has softness and elasticity. Because the silica gel material itself is not easy to adhere to stains or bacteria, it can be cleaned with clean water after daily use. Moreover, the suction nozzle 6300 can remain intact after repeated tensile tests and tear resistance tests, and it also gives infants a comfortable experience.

The suction nozzle 6300 is fixed to the upper compartment 6110 through the suction nozzle mounting seat 6111 provided in the upper compartment 6110. When in use, the suction nozzle 6300 is placed close to the hand palm, and the appropriate suction gear is adjusted by the control button 1520 to perform a nasal aspirator, and the suction nozzle 6300 is placed at the nostril without extending into the nasal cavity. Before using the nasal aspirator capable of automatically relieving pressure, a normal saline can be dripped to soften nasal secretions. After suction, the storage compartment 6100 can be opened and cleaned with clean water.

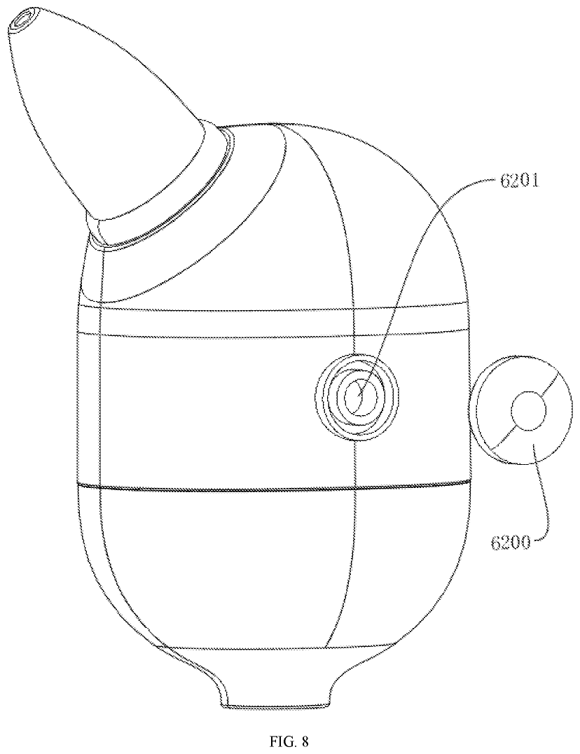

As shown in FIG. 8, FIG. 8 shows another embodiment of the nasal aspirator capable of automatically relieving pressure. The storage compartment 6100 in the suction component 6000 is provided with a pressure releasing channel 6201 and a switch component 6200. The switch component 6200 controls the opening and closing of the pressure releasing channel 6201. When the pressure releasing channel 6201 is closed, a negative pressure space is formed, and the air pump component 2000 works to generate negative pressure to form suction, and after passing through the air bag 3000, the hose 5000, the storage compartment 6100 and the suction nozzle 6300 in turn, suction is generated at the suction nozzle 6300 and communicated with the outside. When the pressure releasing channel 6201 is opened, an additional pressure releasing channel is provided on the storage compartment 6100, which makes the whole nasal aspirator safer.

In another embodiment, the nasal aspirator has a casing 1000 containing a cavity 7000, an air pump component 2000, an air bag 3000, a constant pressure device 4000 and a suction component 6000 arranged in the cavity 7000. The suction component 6000 includes a storage compartment 6100, and a suction nozzle 6300, that is, the suction component 6000 does not include a hose 5000 (not shown in the figure).

In another embodiment, the nasal aspirator includes a casing 1000 with an accommodating cavity 7000, an air pump component 2000, an air bag 3000, a constant pressure device 4000 and a suction component 6000 arranged in the accommodating cavity 7000, wherein the air pump component 2000 includes an air flow connector 2001, and the air bag 3000 includes an air chamber 3001, and the constant pressure device 4000 is arranged on the air bag 3000 and communicates with the air chamber 3001 (not shown in the figure).

In another embodiment, the number of constant pressure devices 4000 is two. In other embodiments, the number of constant pressure devices 4000 can be any desired number.

In another embodiment, the number of air bags 3000 is two, and in other embodiments, the number of air bags 3000 can be any desired number.

In another embodiment, the air suction component 6000 can be used once and does not need to be cleaned after use, thus avoiding the possibility of virus infection due to improper cleaning. Because it is impossible to control whether the user performs the cleaning operation correctly or not, it may become a source of bacteria over a long period of time. However, by providing disposable substitutes, the spread of bacteria can be effectively prevented and the health and safety of users can be guaranteed.

In another embodiment, the nasal aspirator does not include a lighting component 1400 or a music component 1300 (not shown in the figure).

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

The invention has now been described in detail for the purposes of clarity and understanding. However, those skilled in the art will appreciate that certain changes and modifications may be practiced within the scope of the appended claims.
Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

What is claimed is:

1. A nasal aspirator capable of automatically relieving pressure, comprising:
a casing with an accommodating cavity, an air pump component, an air bag, a constant pressure device and a suction component arranged in said accommodating cavity; and
wherein, said air pump component is communicated with said air bag and comprises at least one air flow connector, and said air flow connector defines an air suction channel and an air exhaust channel of said air pump component; and
wherein said air bag comprises at least one air chamber, and at least one communication member is arranged in said air chamber, and said communication member is configured to receive said air flow connector; and
wherein, said constant pressure device is arranged on said air bag and communicated with said air chamber, and comprises at least one pressure releasing valve; and the pressure releasing valve provides an overflow channel for air in said air chamber when the air in said air chamber exceeds a predetermined threshold; and
wherein said suction component is communicated with said air bag, and said suction component defines a path for external air to enter said air bag.

2. The nasal aspirator according to claim 1, wherein said air flow connector comprises an air suction connector and an air exhaust connector; and said air chamber comprises an air suction chamber and an air exhaust chamber, and said communication member comprises an air suction communication member and an air exhaust communication member; and said air suction connector is arranged on said air suction communication member and communicates with said air suction chamber, and said air exhaust connector is arranged on said air exhaust communication member and communicates with said air exhaust chamber.

3. The nasal aspirator according to claim 2, wherein said constant pressure device is communicated with said air suction chamber, and when said pressure in said air suction chamber reaches a threshold, said pressure releasing valve is opened; and when said pressure in said suction chamber is lower than the threshold, said pressure releasing valve is closed.

4. The nasal aspirator according to claim 3, wherein said casing is provided with an air suction port and an air exhaust port communicated with said air bag, wherein said air suction port is communicated with said air suction chamber, and said air exhaust port is communicated with said air exhaust chamber.

5. The nasal aspirator according to claim 1, wherein said air suction component comprises a hose, a storage compartment and a suction nozzle, wherein one end of said storage compartment is communicated with said hose and the other end is communicated with said suction nozzle, and a channel for air to flow is formed between said storage compartment and said hose.

6. The nasal aspirator according to claim 5, wherein said storage compartment comprises an upper compartment, a lower compartment, a sealing ring and a bracket; and said lower compartment is also provided with a slot, and said sealing ring is fixed on said slot.

7. The nasal aspirator according to claim 6, wherein said lower compartment is provided with a mounting seat, which comprises an upper mounting seat and a lower mounting seat, said bracket is fixed on said upper mounting seat, and said hose is fixed on said lower mounting seat.

8. The nasal aspirator according to claim 6, wherein said upper compartment is provided with a suction nozzle mounting seat, and said suction nozzle is fixed on said suction nozzle mounting seat.

9. The nasal aspirator according to claim 7, wherein said bracket comprises a first cover plate, a second cover plate arranged below said first cover plate and an air flow pipe; and wherein that first cover plate and said second cover plate are circular, a diameter of said first cover plate is larger than that of said second cover plate, and a through hole is also arranged on said air flow pipe, and said through hole is positioned between said first cover plate and said second cover plate.

10. The nasal aspirator according to claim 6, wherein at least a part of said storage compartment is made of a transparent material.

11. The nasal aspirator according to claim 1, wherein a battery component, a music playing component and a lighting component are also arranged in said accommodating cavity.

12. The nasal aspirator according to claim 11, wherein an electronic control module is further arranged in said accommodating cavity, and said electronic control module comprises a control circuit board and a control button, wherein said control circuit board is electrically connected to said air pump component, said music playing component and said lighting component, and said control button is arranged on said control circuit board.

13. The nasal aspirator according to claim 12, wherein said nasal aspirator has different suction gears and is controlled by said control button.

14. The nasal aspirator according to claim 12, wherein said casing is provided with a display panel, which is connected to said electronic control module and used for displaying a current working state of said nasal aspirator.

15. A nasal aspirator capable of automatically relieving pressure, comprising:
a casing with an accommodating cavity, an air pump component, an air bag, a constant pressure device and a suction component arranged in said accommodating cavity; and
wherein, said air pump component is communicated with said air bag and comprises at least one air flow connector, and said air flow connector defines an air suction channel and an air exhaust channel of said air pump component; and
wherein said air bag comprises at least one air chamber, and at least one communication member is arranged in said air chamber, and said communication member is configured to receive said air flow connector; and
wherein, said constant pressure device is arranged on said air bag and communicated with said air chamber, and comprises at least one pressure releasing valve; and the pressure releasing valve provides an overflow channel for air in said air chamber when the air in said air chamber exceeds a predetermined threshold; and
wherein, said suction component defines an airflow path for external air to enter said air bag, and comprises a suction nozzle, a storage compartment and a hose, wherein said storage compartment is arranged between said suction nozzle and said hose, said storage compartment is configured to contain nasal mucus, and said hose is communicated with said air bag; and
wherein said storage compartment further comprises a pressure releasing channel and a switch component, and said switch component can open and close said pressure releasing channel.

16. A method for sucking nasal mucus, comprising the following steps: providing a nasal aspirator capable of automatically relieving pressure, wherein said nasal aspirator comprises a casing with an accommodating cavity, an air pump component, an air bag, a constant pressure device and a suction component arranged in said accommodating cavity; and wherein, said air pump component is communicated with said air bag and comprises at least one air flow connector, and said air flow connector defines an air suction channel and an air exhaust channel of said air pump component;
and wherein said air bag comprises at least one air chamber, at least one communication member is arranged in said air chamber, and said communication member is configured to receive said air flow connector; and wherein said constant pressure device is arranged on said air bag and communicated with said air chamber, and comprises at least one pressure releasing valve, and said pressure releasing valve provides an overflow channel for air in said air chamber when the air in said air chamber exceeds a predetermined threshold; and wherein said suction component is communicated with said air bag and comprises a suction nozzle and a storage compartment, and said suction component defines a path for external air to enter said air bag; and
opening said air pump component;
placing said suction nozzle close to a source of nasal mucus;
absorbing said nasal mucus and storing said nasal mucus in said storage compartment; and
closing said air pump component after absorbing said nasal mucus.

17. The method according to claim 16, wherein before placing said suction nozzle near the source of nasal mucus, said suction nozzle is put near a hand palm to adjust an appropriate suction gear before sucking the nasal mucus.

18. The method according to claim 16, wherein when said suction nozzle is placed close to said source of nasal mucus, said suction nozzle is placed at a nostril without going deep into a nasal cavity.

19. The method according to claim 16, wherein before placing said suction nozzle near the source of nasal mucus, a normal saline is used to soften the nasal mucus.

20. The method according to claim 16, wherein after closing said air pump component, said storage compartment is opened and cleaned.
